(19) United States
(12) Patent Application Publication
(10) Pub. No.: US 2016/0244555 A1
(43) Pub. Date: Aug. 25, 2016

(54) PROCESS FOR PRODUCING A STREAM COMPRISING ETHYLENE GLYCOL

(71) Applicant: BIOCHEMTEX S.P.A., Tortona (Alessandria) (IT)

(72) Inventors: Marco BERNARDI, Bologna (IT); Daniela TODARO, Milano (IT); Chiara DORATI, Pontecurone (IT)

(73) Assignee: Biochemtex S.p.A., Tortona, AL (IT)

(21) Appl. No.: 15/025,980

(22) PCT Filed: Oct. 16, 2014
(86) PCT No.: PCT/EP2014/002794

§ 371 Date: Mar. 30, 2016

(30) Foreign Application Priority Data
Oct. 16, 2013 (IT) TO2013A 000833

(51) Int. Cl. C08G 63/183; B01J 21/18; B01J 21/04; C01B 3/32; B01J 23/42; B01J 23/46; B01J 23/755

(52) U.S. Cl. CPC C08G 63/183; B01J 23/462; B01J 21/18; B01J 23/755; C01B 3/326; B01J 23/42; B01J 21/04; C01B 2203/0233; C01B 2203/06

(57) ABSTRACT

It is disclosed a process for producing a low boiling mixture comprising ethylene glycol and propylene glycol from a liquid sugar stream derived from a ligno-cellulosic biomass feedstock. The liquid sugar stream is catalytically converted in the presence of hydrogen to a mixture, which is separated into at least a high boiling mixture, comprising glycerol, and the low boiling mixture. The high boiling mixture is converted to hydrogen by reforming and the reforming hydrogen is used in the catalytical steps. Preferably, all the hydrogen used in the conversion process is generated by aqueous phase reforming of the high boiling polyols mixture.

### BACKGROUND

Conversion of biomass has attracted significant attention as a key technology for replacing oil as the source of renewable fuels and chemicals. Lignocellulose is the most abundant biomass resource, and is not digestible for human beings, which is an advantage over sugars and starch since the use of edible carbohydrates has competed with the food production. Therefore, lignocellulose is one of the most attractive biomass resources in nature, available at a very low cost.

For effectively replacing fossil oil, renewable fuels and chemicals have not only to meet the technical specification in terms of performance, but they must be produced at a competitive cost with the oil derived competitors.

Ethylene glycol and propylene glycol are two oil-derived polyols which are widely used as starting materials in the polymer chemistry. Many processes have been developed for converting water soluble and insoluble sugar sources to polyols. Nevertheless, even if the conversion chemistry is well known, none of prior art processes has been found real industrial applicability so far. Some of the prior art methods demonstrate the conversion of synthetic sugars to light polyols; in the case of conversion of lignocellulose derived sugars, the prior art processes produce polyols mixtures having poor properties for finding real use and in general are too much expensive for competing with oil derived polyols. One of the main issue is related to the fact that it is difficult to control the competitive reaction pathways, therefore the stream derived from the ligno-cellulosic feedstock is a mixture which usually comprises many compounds.

One of the main issue in the conversion of sugars to polyols is represented by the cost of Hydrogen used in the reactions. In real conversion plants, facilities for producing Hydrogen on-site have to be installed. The most economical solution is methane reforming, which is not a source of renewable Hydrogen; water electrolysis is a clean source of Hydrogen, but it is expensive. Another strategy is to locate the polyols conversion plant close to an oil refinery, where Hydrogen is produced in the refining process. Even if at low cost, this solution uses oil derived Hydrogen and real implementation is limited by the availability of refinery sites.

As exemplary prior art of conversion of biomass-derived xylose to xylitol, in H M Baudel et al., “Xylitol production via catalytic hydrogenation of sugarcane bagasse dissolving pulp liquid effluents over Ru/C catalyst”, J. Chem. Technol. Biotechnol. 80:230-233 (2005), xylose-rich liquid effluents generated by the acid hydrolysis of sugarcane bagasse for production of dissolving pulp were converted to xylitol, via catalytic hydrogenation over Ru/C. The paper shows that Ruthenium (Ru 2%/C) catalysts are suitable to convert bagasse hydrolysate sugars into polyols, with high selectivity towards xylitol (above 98%), even under mild temperature and hydrogen pressure levels (80° C., 20 atm).

As exemplary prior art of conversion of xylitol to polyols, in J. Sun et al., “Selective hydrogenolysis of biomass-derived xylitol to ethylene glycol and propylene glycol on supported Ru catalysts”, Green Chem., 2011, 13, p. 135, the selective hydrogenolysis of biomass-derived xylitol to ethylene glycol and propylene glycol was carried out on different catalysts in the presence of Ca(OH)₂. The catalysts included Ru supported on activated carbon (C) and, for comparison, on metal oxides, Al₂O₃, TiO₂, ZrO₂ and Mg₂AlOₓ as well as C-supported other noble metals, Rh, Pd and Pt. The authors show how to control the selectivity of the hydrogenolysis of xylitol to different polyols, comprising ethylene glycol, propylene glycol, glycerol, threitol, arabitol. A relevant amount of lactic acid is also produced as a by-product.

U.S. Pat. No. 6,964,757 discloses a method of producing hydrogen from oxygenated hydrocarbon reactants, such as methanol, glycerol, sugars (e.g. glucose and xylose), or sugar alcohols (e.g. sorbitol). The method takes place in the condensed liquid phase. The method includes the steps of reacting water and a water-soluble oxygenated hydrocarbon in the presence of a metal-containing catalyst. The catalyst contains a metal selected from the group consisting of Group VIIIB transitional metals, alloys thereof, and mixtures thereof. The method can be run at lower temperatures than those used in the conventional steam reforming of alkanes. Other methods are disclosed in U.S. Pat. No. 6,699,457, U.S. Pat. No. 6,953,873 and U.S. Pat. No. 6,964,758.

In US20110313209A1 it is disclosed a catalytic process for generating at least one polyol from a feedstock comprising cellulose. The process involves contacting, continuously, hydrogen, water, and a feedstock comprising cellulose, with a catalyst to generate an effluent stream comprising at least one polyol, water, hydrogen, and at least one co-product. Unreacted hydrogen is recycled to the reaction zone. Co-products may be reaction intermediates which may be separated from the reaction zone effluent and recycled to the reaction zone.

U.S. Pat. No. 8,198,486 discloses methods for generating propylene glycol, ethylene glycol and other polyols. The methods involve reacting a first portion of an aqueous stream of a biomass feedstock solution over a catalyst under aqueous phase reforming conditions to produce hydrogen, and then reacting the hydrogen and a second portion of the aqueous feedstock solution over a catalyst to produce propylene glycol, ethylene glycol and the other polyols. The use of a portion of the feedstock for producing the Hydrogen needed for generating polyols reduces the global yield of the process.

There is therefore the need of an improved process for producing polyols from a biomass feedstock, optimizing the conversion yield of the biomass feedstock to polyols and simultaneously reducing or eliminating the need of external Hydrogen.

### BRIEF DESCRIPTION OF THE INVENTION

It is disclosed a process for producing a low boiling mixture comprising ethylene glycol and propylene glycol, wherein the process comprises the steps of:
a. Hydrogenating a liquid sugar stream derived from a ligno-cellulosic biomass feedstock, said liquid sugar stream comprising water and at least a solubilized monomeric sugar, by contacting the liquid sugar stream with a hydrogenation catalyst in the presence of Hydrogen, at a hydrogenation pressure in the range of 30 bar to 150 bar and at a hydrogenation temperature in the range of 50° C. to 200° C., and for a hydrogenation time sufficient to produce a hydrogenated mixture comprising water and at least a sugar alcohol;
b. Conducting hydrogenolysis of at least a portion of the hydrogenated mixture, by contacting the at least a portion of the hydrogenated mixture with a hydrogenolysis catalyst in the presence of OH ions and Hydrogen, at a hydrogenolysis pressure in the range of 40 bar to 170 bar, at a hydrogenolysis temperature and for a hydrogenolysis time sufficient to produce a hydrogenolysis mixture, comprising ethylene glycol, propylene glycol and glycerol;
c. Separating at least a portion of the hydrogenolysis mixture into at least the low boiling mixture comprising ethylene glycol and propylene glycol, and a high boiling mixture comprising glycerol;
d. Reforming at least a portion of the high boiling mixture, by contacting the at least a portion of the high boiling mixture with a reforming catalyst under reforming conditions and for a reforming time sufficient to produce a reforming gas product comprising reforming Hydrogen,
wherein the Hydrogen of the hydrogenation step and/or the Hydrogen of the hydrogenolysis step comprises at least a portion of the reforming Hydrogen.

It is also disclosed that the percent ratio of the amount of reforming Hydrogen in the hydrogenation step to the total amount of Hydrogen in the hydrogenation step may be greater than a value selected from the group consisting of 10%, 30%, 50%, 60%, 70%, 80%, 90%, and 95%.

It is further disclosed that the percent ratio of the amount of reforming Hydrogen in the hydrogenolysis step to the total amount of Hydrogen in the hydrogenolysis step may be greater than a value selected from the group consisting of 10%, 30%, 50%, 60%, 70%, 80%, 90%, and 95%.

It is also disclosed that the percent amount by weight on a dry matter basis of glycerol in the hydrogenolysis mixture may be a value in a range selected from the group consisting of 5% to 40%, 10% to 30%, and 15% to 20% of the total amounts of polyols in the hydrogenolysis mixture.

It is further disclosed that the hydrogenolysis temperature may be a value in a range selected from the group consisting of 150° C. to 240° C., and 190° C. to 220° C.

It is also disclosed that the hydrogenolysis pressure may be a value in a range selected from the group consisting of 40 bar to 150 bar, 50 bar to 100 bar, and 60 to 80 bar.

It is also disclosed that the molar ratio of the total amount of the sugar alcohols to the amount of Hydrogen in the hydrogenolysis step may be a value in a range selected from the group consisting of 1:2 to 1:10, 1:3 to 1:8, and 1:4: to 1:6.

It is further disclosed that the hydrogenolysis step may occur in a batch mode and the hydrogenolysis time may be a value in a range selected from the group consisting of 10 minutes to 10 hours, 20 minutes to 8 hours, 30 minutes to 7 hours, 45 minutes to 6 hours, 60 minutes to 4 hours, and 90 minutes to 3 hours.

It is also disclosed that the hydrogenolysis step may occur in a continuous or semi-continuous mode having a hydrogenolysis liquid hourly space velocity, and the hydrogenolysis liquid hourly space velocity may be a value in a range selected from the group consisting of 0.1 to 4 h⁻¹ 0.2 to 3 h⁻¹, 0.5 to 2.5 h⁻¹, and 1 to 2 h⁻¹.

It is further disclosed that the hydrogenolysis catalyst may be a supported metal catalyst comprising at least a metal selected from the group consisting of Ru, Ni, Cu and Pt, or combination thereof.

It is also disclosed that the support may comprise at least a compound selected from the group consisting of alumina, zirconia and activated carbon, or a combination thereof.

It is further disclosed that the hydrogenation temperature may be a value in a range selected from the group consisting of 70° C. to 150° C., 85° C. to 130° C., and 100 to 120° C.

It is also disclosed that the hydrogenation pressure may be a value in a range selected from the group consisting of 40 bar to 150 bar, 50 bar to 100 bar, and 60 to 80 bar.

It is further disclosed that the molar ratio of the total amount of solubilized monomeric sugars to the amount of Hydrogen in the hydrogenation step may be a value in a range selected from the group consisting of 1:2 to 1:10, 1:3 to 1:8, and 1:4: to 1:6.

It is also disclosed that the hydrogenation step may occur in a batch mode and the hydrogenation time may be a value in a range selected from the group consisting of 30 minutes to 240 minutes, 45 minutes to 180 minutes, and 60 minutes to 120 minutes.

It is further disclosed that the hydrogenation step may occur in a continuous or semi-continuous mode having a hydrogenation liquid hourly space velocity, and the hydrogenation liquid hourly space velocity may be a value in a range selected from the group consisting of 0.2 to 3 h⁻¹, 0.5 to 2.5 h⁻¹, and 1 to 2 h⁻¹.

It is also disclosed that the hydrogenation catalyst may be a supported metal catalyst comprising at least a metal selected from the group consisting of Ru, Ni and Pt, or combination thereof.

It is further disclosed that the support may comprise at least a compound selected from the group consisting of alumina, zirconia and activated carbon, or a combination thereof.

It is also disclosed that the reforming may be conducted in aqueous phase under aqueous phase reforming conditions.

It is further disclosed that the reforming aqueous phase reforming conditions may comprise a reforming temperature, and the reforming temperature may be a value in a range selected from the group consisting of 100° C. to 400° C., 150° C. to 350° C., and 200° C. to 300° C.

It is also disclosed that the aqueous phase reforming conditions may comprise a reforming pressure, and the reforming pressure may be selected to maintain at least a portion of the high boiling polyols in the high boiling mixture in a liquid state at the reforming temperature.

It is further disclosed that water may added to the high boiling mixture.

It is also disclosed that the reforming catalyst may be a supported metal catalyst comprising at least a metal selected from the group consisting of Pt, Ru, Re, Pd, Rh, Ni and Co, or combination thereof.

It is further disclosed that the support may comprise at least a compound selected from the group consisting of alumina, activated carbon, silica, zeolite, titania, zirconia and ceria, or a combination thereof.

It is also disclosed that the reforming step may occur in a continuous or semi-continuous mode having a reforming liquid hourly space velocity, and the reforming liquid hourly space velocity may be a value in a range selected from the group consisting of 0.1 to 10 h⁻¹, 0.5 to 10 h⁻¹, 1 to 10 h⁻¹, 2 to 10 h⁻¹, 4 to 8 h⁻¹, and 5 to 7 h⁻¹.

It is further disclosed that the steps hydrogenation, hydrogenolysis and reforming may be conducted in three separated vessels.

It is also disclosed that the at least a sugar alcohol may comprise a compound selected from the group consisting of xylitol, sorbitol and arabitol, or mixture thereof.

It is further disclosed that the at least a solubilized monomeric sugar may comprise a compound selected from the group consisting of xylose, glucose and arabinose, or mixture thereof.

It is also disclosed that the at least a solubilized monomeric sugar may comprise xylose and the percent amount of xylose by weight on a dry matter basis in the liquid sugar stream may be greater than a value selected from the group consisting of 50%, 70%, 80%, 90% and 95%.

It is further disclosed that at least a portion of the ethylene glycol and at least a portion of the propylene glycol in the low boiling mixture are separated from the low boiling mixture to produce an ethylene glycol stream comprising ethylene glycol and a propylene glycol stream comprising propylene glycol.

It is also disclosed that the ethylene glycol stream may further comprise at least one diol selected from the group consisting of 1,2-Propylene glycol, 1,2-Butanediol and 1,2-Pentanediol.

It is further disclosed that the ethylene glycol stream may be used for producing a polyester resin.

It is also disclosed that the polyester may comprise acid moieties and at least 85 mole % of the acid moieties may be derived from terephthalic acid or its dimethyl ester.

It is further disclosed that the polyester resin may be used for producing a polyester bottle.

### BRIEF DESCRIPTION OF THE FIGURES

FIG. 1 is a schematic diagram illustrating one embodiment of the disclosed process.

FIG. 2 is a schematic diagram illustrating another embodiment of the disclosed process.

FIG. 3 is a schematic diagram illustrating one embodiment of the process for converting the ligno-cellulosic feedstock to the liquid sugar stream.

### DETAILED DESCRIPTION

The disclosed process relates to the catalytic conversion of a liquid sugar stream to a low boiling mixture comprising ethylene glycol and propylene glycol. The liquid sugar stream is subjected to a hydrogenation reaction in the presence of Hydrogen and an hydrogenating catalyst to produce a hydrogenated mixture comprising one or more sugar alcohols. The hydrogenated mixture is then reacted with hydrogen in the presence of a hydrogenolysis catalyst to produce a hydrogenolysis mixture, comprising ethylene glycol, propylene glycol and glycerol.

According to one aspect of the invention, it is disclosed a process for producing a mixture comprising ethylene glycol and propylene glycol from a ligno-cellulosic feedstock without the need—or with the need of a limited amount—of external hydrogen, which represents an important issue in the economics of the process. Namely, the hydrogenolysis mixture is separated in at least low boiling mixture and a high boiling mixture, preferably by means of thermal processes, such as for instance by means of thermal evaporation. Other streams may be produced in the separation step. In the context of the present disclosure, low boiling compounds are compounds which may be evaporated and recovered in the condensed evaporate when the hydrogenolysis mixture is heated to 120° C. at a pressure of 50 mbar. The low boiling mixture comprises ethylene glycol and propylene glycol, and may further comprise other polyols, such as for instance butanediol, pentanediols, and/or other compounds which are not polyols, such as for instance unreacted or intermediates or byproducts. In the context of the present disclosure, high boiling compounds are compounds which may be recovered in the condensate when the hydrogenolysis mixture is heated to 120° C. at a pressure of 50 mbar. The high boiling mixture comprises glycerol and may further comprise other polyols, such as threitol e I'erythrithol, and/or other compounds which are not polyols, such as for instance lactic acid, formic acid (which in a basic environment may be present in anionic form, such as for instance sodium lactate and sodium formate) and unreacted sugar alcohols, which are not evaporated at the separation conditions.

The high boiling mixture is then converted to Hydrogen under reforming conditions, and the hydrogen is used for supplying the hydrogenation and/or hydrogenolysis reactions. The high boiling mixture is an unavoidable byproduct of the disclosed process for producing the low boiling mixture comprising ethylene glycol and propylene glycol. Thereby its use for generating at least a portion of the hydrogen needed for the catalytic conversion does not affect the yield of the disclosed process. Preferably, reforming is conducted in aqueous liquid phase conditions of the high boiling mixture in a reactor separated from the reactor or reactors of the hydrogenation and hydrogenolysis reaction, for avoiding any contamination of the hydrogenation and hydrogenolysis catalysts by the reforming mixture and by-products. Therefore, according to another aspect of the invention, it is disclosed a process which improves the lifetime of the catalysts.

In a preferred embodiment, the liquid sugar stream is derived from the ligno-cellulosic feedstock by means of an inexpensive soaking in water, which solubilize a portion of the carbohydrates in the feedstock without the need of chemicals or catalysts. The solid, not solubilized feedstock may then be used for feeding another conversion process or processes to different chemical end-products. The soaking of the ligno-cellulosic feedstock produces a soaked liquid stream comprising soluble sugars, mainly derived from the xylans of the ligno-cellulosic feedstock, which may be hydrolyzed to monomers and purified before feeding the process of the present disclosure.

Therefore, according to a further aspect of the invention, it is disclosed a process which converts with a low cost liquid sugar stream to valuable chemicals reducing the conversion costs.

### Ligno-Cellulosic Feedstock

In general, a ligno-cellulosic feedstock, indicated also as ligno-cellulosic biomass can be described as follows:

Apart from starch, the three major constituents in plant biomass are cellulose, hemicellulose and lignin, which are commonly referred to by the generic term lignocellulose. Polysaccharide-containing biomasses as a generic term includes both starch and ligno-cellulosic biomasses. Therefore, some types of feedstocks can be plant biomass, polysaccharide containing biomass, and ligno-cellulosic biomass which may or may not contain starch.

Polysaccharide-containing biomasses according to the present invention include any material containing polymeric sugars e.g. in the form of starch as well as refined starch, cellulose and hemicellulose.

Relevant types of ligno-cellulosic feedstock for deriving the claimed invention may include biomasses derived from agricultural crops selected from the group consisting of starch containing grains, refined starch; corn stover, bagasse, straw e.g. from rice, wheat, rye, oat, barley, rape, sorghum; softwood e.g. Pinus sylvestris, Pinus radiate; hardwood e.g. Salix spp. Eucalyptus spp.; tubers e.g. beet, potato; cereals from e.g. rice, wheat, rye, oat, barley, rape, sorghum and corn; waste paper, fiber fractions from biogas processing, manure, residues from oil palm processing, municipal solid waste or the like. Although the experiments are limited to a few examples of the enumerated list above, the invention is believed applicable to all the member of the list.

In one embodiment, the ligno-cellulosic biomass feedstock used in the process is from the family usually called grasses. The proper name is the family known as Poaceae or Gramineae in the Class Liliopsida (the monocots) of the flowering plants. Plants of this family are usually called grasses, or, to distinguish them from other graminoids, true grasses. Bamboo is also included. There are about 600 genera and some 9,000-10,000 or more species of grasses (Kew Index of World Grass Species).

Poaceae includes the staple food grains and cereal crops grown around the world, lawn and forage grasses, and bamboo. Poaceae generally have hollow stems called culms, which are plugged (solid) at intervals called nodes, the points along the culm at which leaves arise. Grass leaves are usually alternate, distichous (in one plane) or rarely spiral, and parallel-veined. Each leaf is differentiated into a lower sheath which hugs the stem for a distance and a blade with margins The leaf blades of many grasses are hardened with silica phytoliths, which helps discourage grazing animals. In some grasses (such as sword grass) this makes the edges of the grass blades sharp enough to cut human skin. A membranous appendage or fringe of hairs, called the ligule, lies at the junction between sheath and blade, preventing water or insects from penetrating into the sheath.

Grass blades grow at the base of the blade and not from elongated stem tips. This low growth point evolved in response to grazing animals and allows grasses to be grazed or mown regularly without severe damage to the plant.

Flowers of Poaceae are characteristically arranged in spikelets, each spikelet having one or more florets (the spikelets are further grouped into panicles or spikes). A spikelet consists of two (or sometimes fewer) bracts at the base, called glumes, followed by one or more florets. A floret consists of the flower surrounded by two bracts called the lemma (the external one) and the palea (the internal). The flowers are usually hermaphroditic (maize, monoecious, is an exception) and pollination is almost always anemophilous. The perianth is reduced to two scales, called lodicules, that expand and contract to spread the lemma and palea; these are generally interpreted to be modified sepals.

The fruit of Poaceae is a caryopsis in which the seed coat is fused to the fruit wall and thus, not separable from it (as in a maize kernel).

There are three general classifications of growth habit present in grasses; bunch-type (also called caespitose), stoloniferous and rhizomatous.

The success of the grasses lies in part in their morphology and growth processes, and in part in their physiological diversity. Most of the grasses divide into two physiological groups, using the C3 and C4 photosynthetic pathways for carbon fixation. The C4 grasses have a photosynthetic pathway linked to specialized Kranz leaf anatomy that particularly adapts them to hot climates and an atmosphere low in carbon dioxide.

C3 grasses are referred to as “cool season grasses” while C4 plants are considered “warm season grasses”. Grasses may be either annual or perennial. Examples of annual cool season are wheat, rye, annual bluegrass (annual meadowgrass, Poa annua and oat). Examples of perennial cool season are orchard grass (cocksfoot, Dactylis glomerata), fescue (Festuca spp), Kentucky Bluegrass and perennial ryegrass (Lolium perenne). Examples of annual warm season are corn, sudangrass and pearl millet. Examples of Perennial Warm Season are big bluestem, indian grass, bermuda grass and switch grass.

One classification of the grass family recognizes twelve subfamilies: These are 1) anomochlooideae, a small lineage of broad-leaved grasses that includes two genera (Anomochloa, Streptochaeta); 2) Pharoideae, a small lineage of grasses that includes three genera, including Pharus and Leptaspis; 3) Puelioideae a small lineage that includes the African genus Puelia; 4) Pooideae which includes wheat, barley, oats, brome-grass (Bronnus) and reed-grasses (Calamagrostis); 5) Bambusoideae which includes bamboo; 6) Ehrhartoideae, which includes rice, and wild rice; 7) Arundinoideae, which includes the giant reed and common reed; 8) Centothecoideae, a small subfamily of 11 genera that is sometimes included in Panicoideae; 9) Chloridoideae including the lovegrasses (Eragrostis, ca. 350 species, including teff), dropseeds (Sporobolus, some 160 species), finger millet (Eleusine coracana (L.) Gaertn.), and the muhly grasses (Muhlenbergia, ca. 175 species); 10) Panicoideae including panic grass, maize, sorghum, sugar cane, most millets, fonio and bluestem grasses; 11) Micrairoideae and 12) Danthoniodieae including pampas grass; with Poa which is a genus of about 500 species of grasses, native to the temperate regions of both hemispheres.

Agricultural grasses grown for their edible seeds are called cereals. Three common cereals are rice, wheat and maize (corn). Of all crops, 70% are grasses.

Sugarcane is the major source of sugar production. Grasses are used for construction. Scaffolding made from bamboo is able to withstand typhoon force winds that would break steel scaffolding. Larger bamboos and Arundo donax have stout culms that can be used in a manner similar to timber, and grass roots stabilize the sod of sod houses. Arundo is used to make reeds for woodwind instruments, and bamboo is used for innumerable implements.

Another ligno-cellulosic biomass feedstock may be woody plants or woods. A woody plant is a plant that uses wood as its structural tissue. These are typically perennial plants whose stems and larger roots are reinforced with wood produced adjacent to the vascular tissues. The main stem, larger branches, and roots of these plants are usually covered by a layer of thickened bark. Woody plants are usually either trees, shrubs, or lianas. Wood is a structural cellular adaptation that allows woody plants to grow from above ground stems year after year, thus making some woody plants the largest and tallest plants.

These plants need a vascular system to move water and nutrients from the roots to the leaves (xylem) and to move sugars from the leaves to the rest of the plant (phloem). There are two kinds of xylem: primary that is formed during primary growth from procambium and secondary xylem that is formed during secondary growth from vascular cambium.

What is usually called “wood” is the secondary xylem of such plants.

The two main groups in which secondary xylem can be found are:

1) conifers (Coniferae): there are some six hundred species of conifers. All species have secondary xylem, which is relatively uniform in structure throughout this group. Many conifers become tall trees: the secondary xylem of such trees is marketed as softwood.
2) angiosperms (Angiospermae): there are some quarter of a million to four hundred thousand species of angiosperms. Within this group secondary xylem has not been found in the monocots (e.g. Poaceae). Many non-monocot angiosperms become trees, and the secondary xylem of these is marketed as hardwood.

The term softwood is used to describe wood from trees that belong to gymnosperms. The gymnosperms are plants with naked seeds not enclosed in an ovary. These seed “fruits” are considered more primitive than hardwoods. Softwood trees are usually evergreen, bear cones, and have needles or scale like leaves. They include conifer species e.g. pine, spruces, firs, and cedars. Wood hardness varies among the conifer species.

The term hardwood is used to describe wood from trees that belong to the angiosperm family. Angiosperms are plants with ovules enclosed for protection in an ovary. When fertilized, these ovules develop into seeds. The hardwood trees are usually broad-leaved; in temperate and boreal latitudes they are mostly deciduous, but in tropics and subtropics mostly evergreen. These leaves can be either simple (single blades) or they can be compound with leaflets attached to a leaf stem. Although variable in shape all hardwood leaves have a distinct network of fine veins. The hardwood plants include e.g. Aspen, Birch, Cherry, Maple, Oak and Teak.

Therefore, in one embodiment, a suitable ligno-cellulosic biomass may be selected from the group consisting of the grasses and woods. In one embodiment, ligno-cellulosic biomass can be selected from the group consisting of the plants belonging to the conifers, angiosperms, Poaceae and families. In one embodiment, Another preferred ligno-cellulosic biomass may be that biomass having at least 10% by weight of it dry matter as cellulose, or more preferably at least 5% by weight of its dry matter as cellulose.

Liquid Sugar Stream Derived from the Ligno-Cellulosic Feedstock

The liquid sugar stream is derived from the ligno-cellulosic feedstock by means of a treatment, or pre-treatment, of the ligno-cellulosic feedstock.

The pre-treatment of the ligno-cellulosic biomass is used to solubilize and remove carbohydrates, mainly xylans and glucans, from the ligno-cellulosic feedstock, and at the same time the concentrations of harmful inhibitory by-products such as acetic acid, furfural and hydroxymethyl furfural remain substantially low.

Pre-treatment techniques which may be used are well known in the art and include physical, chemical, and biological pre-treatment, or any combination thereof. In preferred embodiments the pre-treatment of ligno-cellulosic biomass is carried out as a batch or continuous process.

Physical pre-treatment techniques include various types of milling/comminution (reduction of particle size), irradiation

Comminution includes dry, wet and vibratory ball milling.

Although not needed or preferred, chemical pre-treatment techniques include acid, dilute acid, base, organic solvent, lime, ammonia, sulfur dioxide, carbon dioxide, pH-controlled hydrothermolysis, wet oxidation and solvent treatment.

If the chemical treatment process is an acid treatment process, it is more preferably, a continuous dilute or mild acid treatment, such as treatment with sulfuric acid, or another organic acid, such as acetic acid, citric acid, tartaric acid, succinic acid, or any mixture thereof. Other acids may also be used. Mild acid treatment means at least in the context of the invention that the treatment pH lies in the range from 1 to 5, preferably 1 to 3.

In a specific embodiment the acid concentration is in the range from 0.1 to 2.0% wt acid, preferably sulfuric acid. The acid is mixed or contacted with the ligno-cellulosic biomass and the mixture is held at a temperature in the range of around 160-220° C. for a period ranging from minutes to seconds. Specifically the pre-treatment conditions may be the following: 165-183° C., 3-12 minutes, 0.5-1.4% (w/w) acid concentration, 15-25, preferably around 20% (w/w) total solids concentration. Other contemplated methods are described in U.S. Pat. Nos. 4,880,473, 5,366,558, 5,188,673, 5,705,369 and 6,228,177.

Wet oxidation techniques involve the use of oxidizing agents, such as sulfite based oxidizing agents and the like. Examples of solvent treatments include treatment with DMSO (Dimethyl Sulfoxide) and the like. Chemical treatment processes are generally carried out for about 5 to about 10 minutes, but may be carried out for shorter or longer periods of time.

In an embodiment both chemical and physical pre-treatment is carried out including, for example, both mild acid treatment and high temperature and pressure treatment. The chemical and physical treatment may be carried out sequentially or simultaneously.

The current strategies of thermal treatment are subjecting the ligno-cellulosic material to temperatures between 110-250° C. for 1-60 min e.g.:

Hot water extraction
Multistage dilute acid hydrolysis, which removes dissolved material before inhibitory substances are formed
Dilute acid hydrolysis at relatively low severity conditions
Alkaline wet oxidation
Steam explosion

Almost any pre-treatment with subsequent detoxification.

If a hydrothermal pre-treatment is chosen, the following conditions are preferred:

Pre-treatment temperature: 110-250° C., preferably 120-240° C., more preferably 130-230° C., more preferably 140-220° C., more preferably 150-210° C., more preferably 160-200° C., even more preferably 170-200° C. or most preferably 180-200° C.

Pre-treatment time: 1-60 min, preferably 2-55 min, more preferably 3-50 min, more preferably 4-45 min, more preferably 5-40 min, more preferably 5-35 min, more preferably 5-30 min, more preferably 5-25 min, more preferably 5-20 min and most preferably 5-15 min.

Dry matter content after pre-treatment is preferably at least 20% (w/w). Other preferable higher limits are contemplated as the amount of biomass to water in the pre-treated ligno-cellulosic feedstock be in the ratio ranges of 1:4 to 9:1; 1.3.9 to 9:1, 1:3.5 to 9:1, 1:3.25 to 9:1, 1:3 to 9:1, 1:2.9 to 9:1, 1:2 to 9:1, 1.15 to 9:1, 1:1 to 9:1, and 1:0.9 to 9:1.

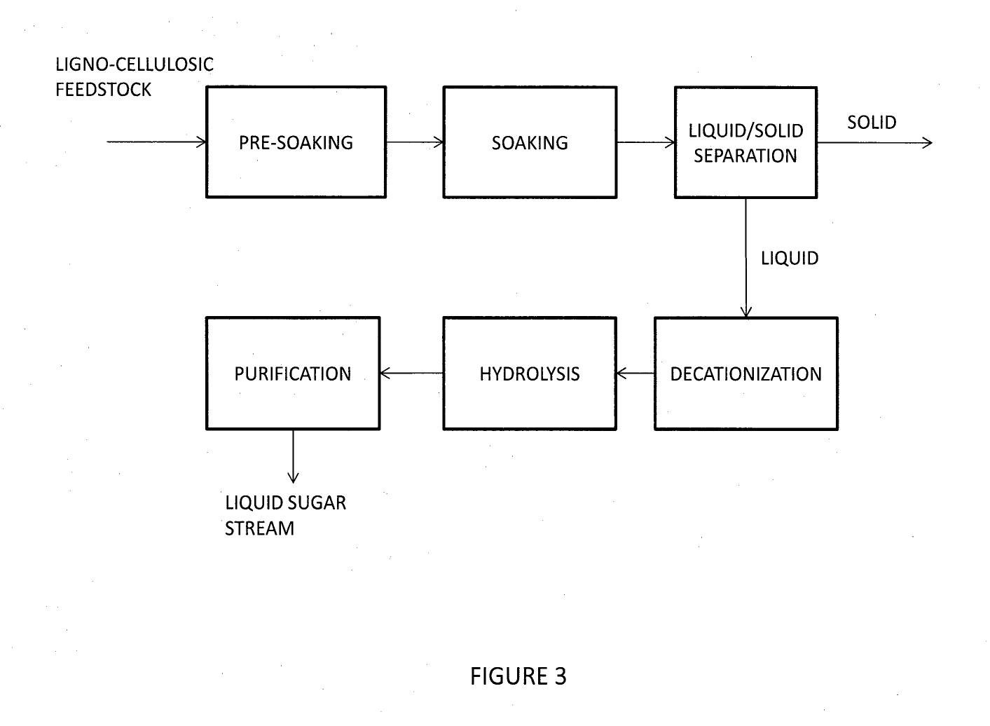

A preferred embodiment of the process used for deriving the liquid sugar stream from the ligno-cellulosic biomass is depicted in FIG. 3 and comprises a pre-treatment and other process steps. A preferred pretreatment of a ligno-cellulosic biomass include a soaking of the ligno-cellulosic biomass feedstock and optionally a steam explosion of at least a part of the soaked ligno-cellulosic biomass feedstock.

The soaking occurs in a substance such as water in either vapor form, steam, or liquid form or liquid and steam together, to produce a product. The product is a soaked biomass containing a soaking liquid, with the soaking liquid usually being water in its liquid or vapor form or some mixture.

This soaking can be done by any number of techniques that expose a substance to water, which could be steam or liquid or mixture of steam and water, or, more in general, to water at high temperature and high pressure. The temperature should be in one of the following ranges: 145 to 165° C., 120 to 210° C., 140 to 210° C., 150 to 200° C., 155 to 185° C., 160 to 180° C. Although the time could be lengthy, such as up to but less than 24 hours, or less than 16 hours, or less than 12 hours, or less than 9 hours or less than 6 hours; the time of exposure is preferably quite short, ranging from 1 minute to 6 hours, from 1 minute to 4 hours, from 1 minute to 3 hours, from 1 minute to 2.5 hours, more preferably 5 minutes to 1.5 hours, 5 minutes to 1 hour, 15 minutes to 1 hour.

If steam is used, it is preferably saturated, but could be superheated. The soaking step can be batch or continuous, with or without stirring. A low temperature soak prior to the high temperature soak can be used. The temperature of the low temperature soak is in the range of 25 to 90° C. Although the time could be lengthy, such as up to but less than 24 hours, or less than 16 hours, or less than 12 hours, or less than 9 hours or less than 6 hours; the time of exposure is preferably quite short, ranging from 1 minute to 6 hours, from 1 minute to 4 hours, from 1 minute to 3 hours, from 1 minute to 2.5 hours, more preferably 5 minutes to 1.5 hours, 5 minutes to 1 hour, 15 minutes to 1 hour.

Either soaking step could also include the addition of other compounds, e.g. H2SO4, NH3, in order to achieve higher performance later on in the process.

The product comprising the soaking liquid, or soaked liquid, is then passed to a separation step where at least a portion of the soaking liquid is separated from the soaked biomass. The liquid will not completely separate so that at least a portion of the soaking liquid is separated, with preferably as much soaking liquid as possible in an economic time frame. The liquid from this separation step is known as the soaked liquid stream comprising the soaking liquid. The soaked liquid will be the liquid used in the soaking, generally water and the soluble species of the feedstock. These water soluble species comprise glucan, xylan, galactan, arabinan, and their monomers and oligomers. The solid biomass is called the first solid stream as it contains most, if not all, of the solids.

The separation of the soaked liquid can again be done by known techniques and likely some which have yet been invented. A preferred piece of equipment is a press, as a press will generate a liquid under high pressure.

The first solid stream may then optionally be steam exploded to create a steam exploded stream, comprising solids. Steam explosion is a well-known technique in the biomass field and any of the systems available today and in the future are believed suitable for this step. The severity of the steam explosion is known in the literature as Ro, and is a function of time and temperature and is expressed as

Ro=texp[(T−100)/14.75]

with temperature, T expressed in Celsius and time, t, expressed in minutes.

The formula is also expressed as Log(Ro), namely

Log(Ro)=Ln(t)+[(T−100)/14.75].

Log(Ro) is preferably in the ranges of 2.8 to 5.3, 3 to 5.3, 3 to 5.0 and 3 to 4.3.

The steam exploded stream may be optionally washed at least with water and there may be other additives used as well. It is conceivable that another liquid may be used in the future, so water is not believed to be absolutely essential. At this point, water is the preferred liquid. The liquid effluent from the optional wash may be added to the soaked liquid stream. This wash step is not considered essential and is optional.

The washed exploded stream is then processed to remove at least a portion of the liquid in the washed exploded material. This separation step is also optional. The term at least a portion is removed, is to remind one that while removal of as much liquid as possible is desirable (preferably by pressing), it is unlikely that 100% removal is possible. In any event, 100% removal of the water is not desirable since water is needed for the subsequent hydrolysis reaction. The preferred process for this step is again a press, but other known techniques and those not invented yet are believed to be suitable. The liquid products separated from this process may be added to the soaked liquid stream.

In an embodiment, the ligno-cellulosic biomass is exposed to a presoaking step before a soaking step in a temperature range of between 10° C. and 150° C., 25° C. to 150° C. even more preferable, with 25° C. to 145° C. even more preferable, and 25° C. to 100° C. and 25° C. to 90° C. also being preferred ranges.

The pre-soaking time could be lengthy, such as up to but preferably less than 48 hours, or less than 24 hours, or less than 16 hours, or less than 12 hours, or less than 9 hours or less than 6 hours; the time of exposure is preferably quite short, ranging from 1 minute to 6 hours, from 1 minute to 4 hours, from 1 minute to 3 hours, from 1 minute to 2.5 hours, more preferably 5 minutes to 1.5 hours, 5 minutes to 1 hour, 15 minutes to 1 hour.

The pre-soaking step is done in the presence of a liquid which is the pre-soaked liquid. After soaking, this liquid preferably has removed less than 5% by weight of the total sugars in the raw material, more preferably, less than 2.5% by weight of the total sugars in the raw-material being more preferable, with less than 1% by weight of the total sugars in the raw material, being the most preferred.

This pre-soaking step is useful as a modification to the soaking step of a biomass pre-treatment step. In soaking (not pre-soaking) of the biomass pre-treatment steps, the soaked liquid stream which has been separated from the soaked solids will preferably have reduced filter plugging components so that the soaked liquid can be easily purified, preferably by means of at least one technique selected from the group of chromatography, nanofiltration and ultrafiltration. Even if in FIG. 3 a single purification stage as last step of the process for deriving the liquid sugar stream from the ligno-cellulosic biomass is represented, the soaked liquid stream may be subjected to more than one purification step, which may be done before hydrolysis or decationization.

The soaked liquid stream will comprise water, sugars which includes monomeric sugars and oligomeric sugars, salts which are dissociated into anions and cations in the soaked liquid stream, optionally phenols, furfural, oils and acetic acid. The soaked liquid stream will in particular contain xylooligomers.

Ideally, the concentration of the total sugars in the soaked liquid stream should be in the range of 0.1 to 300 g/l, with 50 to 290 g/I being most preferred, and 75 to 280 g/I even more preferred, with 100 to 250 g/I most preferred. This concentration can be done by the removal of water. A 50% removal of water increases the concentration of the non-water species by two. While various concentration increases are acceptable, in one embodiment, at least a two fold increase in the concentration of the xyloligomers in the soaked liquid stream is reached. In one embodiment, at least a fourfold increase in the concentration of the xyloligomers in the soaked liquid stream is reached. In one embodiment, at least a six fold increase in the concentration of the xyloligomers in the soaked liquid stream is reached. Many concentration steps may be applied to the soaked liquid stream before or after each process step exemplary depicted in FIG. 3.

In a preferred embodiment, the soaked liquid stream is subjected to hydrolysis for converting at least a portion of the oligomers in the soaked liquid stream to monomers. Hydrolysis of oligomers may be obtained by contacting the soaked liquid stream with a hydrolysis catalyst at hydrolysis conditions. The hydrolysis catalyst may be an inorganic acid, such as sulfuric acid, or an enzyme or enzyme cocktail. The hydrolysis conditions will vary according to the selected hydrolysis catalyst, and are well known in the art.

A preferred way to conduct the hydrolysis of the soaked liquid stream comprises at least two steps, according to the teaching of WO2013026849. The first step is to create an acidic stream from the soaked liquid stream. This is accomplished by increasing the amount of H+ ions to the soaked liquid stream to create the acidic stream. After the desired pH is obtained, the next step is hydrolyzing the oligosaccharides in the acidic stream by raising the temperature of the acidic stream to a hydrolysis temperature for the hydrolysis reaction to occur creating a hydrolyzed stream.

While the creation of the acidic stream can be done in any manner which increases the concentration of H+ ions, a preferred embodiment is to take advantage of the salt content of the soaked liquid stream. In order to obtain the required acidity for the hydrolysis step, the content of salts in the soaked liquid stream can be reduced via cation exchange while at the same time replacing the cations with H+ ions. While the salts may naturally occur in the soaked liquid stream, they can also be added as part of the pre-treatment processes or prior to or during the creation of the acidic stream.

In one embodiment, the hydrolyzed stream is a cleaner liquid, containing almost exclusively monomeric sugars, low content of salts and low amount of degradation products that could hinder subsequent chemical or biological transformations of the sugars.

In a preferred embodiment, the liquid sugar stream comprises at least a portion of the hydrolyzed stream.

In another preferred embodiment, the liquid sugar stream is comprised of at least a portion of the hydrolyzed stream.

### Catalytic Conversion of the Liquid Sugar Stream to Polyols

The liquid sugar stream is converted to a mixture comprising polyols in a catalytic reaction comprising at least two steps, both steps being conducted in the presence of hydrogen.

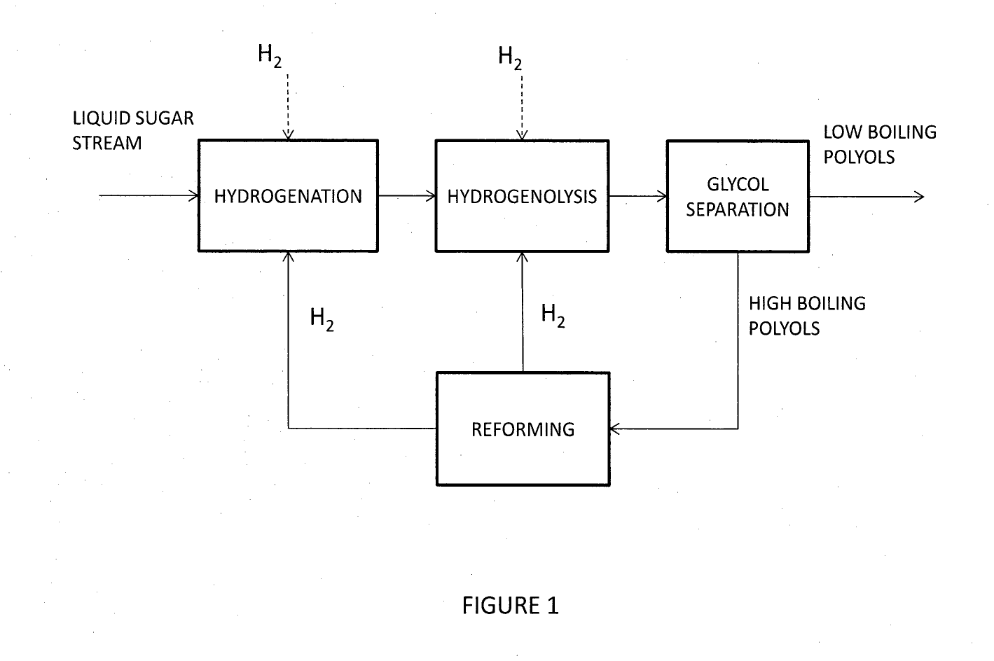

FIG. 1 depicts an exemplary embodiment of the disclosed process. The liquid sugar stream is inserted in a hydrogenation reactor and contacted with a hydrogenation catalyst and hydrogen at hydrogenating conditions promoting the hydrogenation of the sugars in the liquid sugar stream. The hydrogenation catalyst is preferably a supported metal which comprises at least a metal selected from the group of Ru, Ni and Pt, or combination thereof. The catalyst support may comprise alumina, zirconia or activated carbon, or a combination thereof. The ratio between the total amount of the sugars in the liquid sugars stream to the amount of hydrogenation catalyst is preferably between 3:2 and 3:0.5.

Preferably, the hydrogenation reaction is conducted at a hydrogenation temperature promoting the conversion of all, or substantially all, the sugars in the liquid sugar stream. The hydrogenating temperature is between 50° C. to 200° C., preferably between 70° C. to 150° C., more preferably between 85° C. to 130° C., and most preferably between 100 to 120° C.

The hydrogenation reaction may be conducted in a batch mode and for a hydrogenation time sufficient for converting all, or substantially all, the sugars in the liquid sugar stream. The hydrogenation time is preferably between 30 minutes to 240 minutes, more preferably between 45 minutes to 180 minutes, even more preferably between 60 minutes to 120 minutes. The catalyst is preferably present in particle form and dispersed in the liquid sugar stream to effectively promoting the hydrogenation reaction. The content of the hydrogenation reactor may be stirred during the reaction.

In another embodiment, the hydrogenation reaction is conducted in a continuous or semi-continuous mode, wherein the liquid sugar stream is inserted in the hydrogenation reactor and/or the hydrogenated mixture is removed from the reactor continuously or semi-continuously. The continuous or semi-continuous hydrogenation reaction may be characterized by a hydrogenation liquid hourly space velocity of 0.2 to 3 h⁻¹, preferably of 0.5 to 2.5 h⁻¹, most preferably of 1 to 2 h⁻¹. The continuous or semi-continuous hydrogenation reaction may be conducted in a CSTR reactor, the catalyst being preferably present in particle form and dispersed in the liquid sugar stream in the presence of mechanical agitation. A preferred continuous or semi-continuous hydrogenation configuration the fixed bed reactor, even if the hydrogenation reaction may be conducted also in a fluidized bed reactor. The liquid sugar stream, the hydrogenation catalyst and the hydrogen may be introduced in the hydrogenation reactor separately from different inlets or may be premixed before the insertion in the reactor.

The hydrogenation reaction is conducted in the presence of hydrogen, at a hydrogenation pressure in the range of 30 bar to 150 bar, preferably in the range of 40 bar to 150 bar, more preferably in the range of 50 bar to 100 bar, most preferably in the range of 60 bar to 80 bar. In the case of hydrogenation conducted in a batch mode, the hydrogenation pressure corresponds to the pressure at which hydrogen is introduced in the reactor, at the temperature of 25° C. Experimentally, it is measured by a gauge pressure placed on the hydrogen gas supply line, close to the inlet of the hydrogenation reactor and immediately before sealing the reactor. The actual reaction pressure in the reactor may be different from the hydrogenation pressure due to the temperature effect and to contributions of gas reaction products and vapor pressure of the liquid sugar stream at the hydrogenation temperature. In the case of hydrogenation conducted in semi-continuous or continuous mode, wherein is the hydrogen gas flow to be regulated in order to control the pressure inside the reactor, the hydrogenation pressure is the actual pressure inside the reactor at the reaction temperature.

Preferably, the hydrogen and the liquid sugar stream are introduced in the hydrogenation reactor in suitable amounts to reach a molar ratio of the total amount of solubilized monomeric sugar to the hydrogen amount in a range of 1:2 to 1:10, more preferably of 1:3 to 1:8, and most preferably of 1:4: to 1:6. Because the reaction preferably occurs in an stoichiometric excess of Hydrogen for effectively promoting the hydrogenation reaction, a portion of the hydrogen will not react and may be recycled at the end of the reaction and reused in the whole conversion process. In the case of batch reaction, the total amount of hydrogen and the total amount of liquid sugar stream are introduced in the reactor which is then sealed. In the case of continuous or semi-continuous mode, the hydrogen and the liquid sugar stream are introduced in a continuous or semi-continuous way, preferably according the disclosed ranges.

In a preferred embodiment the liquid sugar stream comprise xylose, glucose and arabinose, or mixture, and the hydrogenation reaction of the sugars produces an hydrogenated mixture comprising at least a sugar alcohol. Preferred sugar alcohols are xylitol, sorbitol and arabitol, or mixture thereof.

In a preferred embodiment, the liquid sugar stream is derived from the ligno-cellulosic feedstock by solubilizing mainly the xylans of the ligno-cellulosic feedstock. Thereby, the sugars in the liquid sugar stream comprise mainly xylose and the preferred amount of xylose in the liquid sugar stream on a dry basis is greater than 50%, more preferably greater than 70%, even more preferably greater than 80%, yet even more preferably greater than 90%, being greater than 95% the most preferred value. The corresponding hydrogenated mixture will comprise mainly xylitol and the preferred amount of xylitol in the hydrogenated mixture on a dry basis is greater than 45%, more preferably greater than 70%, even more preferably greater than 80%, yet even more preferably greater than 90%, being greater than 95% the most preferred value.

The hydrogenated mixture is then removed from the hydrogenation reactor and inserted in the hydrogenolysis reactor. If the hydrogenation catalyst is present in dispersed particle form, it is at least in part removed from the reactor together with the hydrogenated mixture, and it may be recovered for instance by means of filtration and reinserted in the hydrogenation reactor, eventually after being regenerated. Eventually, also unwanted hydrogenation products may be removed from the hydrogenated mixture.

The hydrogenated mixture is inserted in a hydrogenolysis reactor and contacted with a hydrogenolysis catalyst and hydrogen at hydrogenolysis conditions promoting the hydrogenolysis of the sugar alcohols in the hydrogenated mixture. The hydrogenolysis catalyst comprises preferably a supported metal selected from the group of Ru, Ni and Pt, or combination thereof. The catalyst support may comprise alumina, zirconia or activated carbon, or a combination thereof. The ratio between the total amount of the alcohols in the hydrogenated mixture to the amount of hydrogenolysis catalyst is preferably between 3:2 and 3:0.1.

The hydrogenolysis reaction of the sugar alcohols occurs in the presence of OH⁻ ions which affects the pH of the reaction environment. pH values greater than 9, corresponding to basic conditions, promote the effective hydrogenolysis of the sugar alcohols. OH⁻ ions are preferably derived from a compound selected from the group consisting of NaOH, KOH, Ca(OH)₂ and Ba(OH)₂, or a combination thereof. The source of OH⁻ ions may be introduced in the hydrogenolysis reactor or it may be added to the hydrogenated mixture before the insertion in the reactor.

The hydrogenolysis reaction is conducted at a hydrogenolysis temperature promoting the conversion of the alcohols in the hydrogenated mixture. The hydrogenolysis temperature may be a value between 150° C. to 240° C., and most preferably between 190 to 220° C.

The hydrogenolysis reaction may be conducted in a batch mode and for a hydrogenolysis time which is preferably sufficient for converting all, or substantially all, the sugar alcohols in the hydrogenated mixture. The hydrogenolysis time is preferably between 10 minutes to 10 hours, more preferably between 20 minutes to 8 hours, even more preferably between 30 minutes to 7 hours, yet even more preferably between 45 minutes to 6 hours, most preferably between 60 minutes to 4 hours, and even most preferably between 90 minutes to 3 hours. The hydrogenolysis catalyst is preferably present in particle form and dispersed in the hydrogenated mixture to effectively promoting the hydrogenolysis reaction. The content of the hydrogenolysis reactor may be stirred during the reaction.

In another embodiment, the hydrogenolysis reaction is conducted in a continuous or semi-continuous mode, wherein the hydrogenated mixture is inserted in the hydrogenation reactor and/or the hydrogenolysis mixture is removed from the reactor continuously or semi-continuously. The continuous or semi-continuous hydrogenolysis reaction may be characterized by a hydrogenolysis liquid hourly space velocity of 0.1 to 4 h⁻¹, preferably of 0.2 to 3 h⁻¹, more preferably of 0.5 to 2.5 h⁻¹ and most preferably of 1 to 2 h⁻¹. The continuous or semi-continuous hydrogenolysis reaction may be conducted in a CSTR reactor, the hydrogenolysis catalyst being preferably present in particle form and dispersed in the hydrogenated mixture in the presence of mechanical agitation. A preferred continuous or semi-continuous hydrogenolysis configuration is the fixed bed reactor, even if the hydrogenolysis reaction may be conducted also in a fluidized bed reactor. The hydrogenated mixture, the hydrogenolysis catalyst and the hydrogen may be introduced in the hydrogenolysis reactor separately from different inlets or may be premixed before the insertion in the reactor.

The hydrogenolysis reaction is conducted in the presence of hydrogen of a hydrogen, at a hydrogenolysis pressure in the range of 40 bar to 170 bar, preferably in the range of 40 bar to 150 bar, more preferably in the range of 50 bar to 100 bar, most preferably in the range of 60 bar to 80 bar. In the case of hydrogenolysis conducted in a batch mode, the hydrogenolysis pressure corresponds to the pressure at which hydrogen is introduced in the reactor, at the temperature of 25° C. Experimentally, it is measured by a gauge pressure placed on the hydrogen gas supply line, close to the inlet of the hydrogenolysis reactor and immediately before sealing the reactor. The actual reaction pressure in the reactor may be different from the hydrogenolysis pressure due to the temperature effect and to contributions of gas reaction products and vapor pressure of the liquids at the hydrogenolysis temperature. In the case of hydrogenolysis conducted in semi-continuous or continuous mode, wherein is the hydrogen gas flow to be regulated in order to control the pressure inside the reactor, the hydrogenolysis pressure is the actual pressure inside the reactor at the reaction temperature.

Preferably, the hydrogen and the hydrogenated mixture are introduced in the hydrogenolysis reactor in suitable amounts to have a molar ratio of the total amount of sugar alcohols to the hydrogen amount in a range of 1:2 to 1:10, more preferably of 1:3 to 1:8, and most preferably of 1:4: to 1:6. Because the reaction preferably occurs in a stoichiometric excess of Hydrogen for effectively promoting the hydrogenolysis reaction, a portion of the hydrogen will not react and may be recycled at the end of the reaction and reused in the whole conversion process. In the case of batch reaction, the total amount of hydrogen and the total amount of hydrogenated mixture are introduced in the reactor which is then sealed. In the case of continuous or semi-continuous mode, the hydrogen and the hydrogenated mixture are introduced in a continuous or semi-continuous way, preferably according the disclosed ranges.

The hydrogenolysis reaction of the sugar alcohols in the hydrogenated mixture produces a hydrogenolysis mixture comprising water ethylene glycol, propylene glycol and glycerol. It may further comprise other polyols, unwanted compounds, comprising acid lactic or formic acid, and unreacted sugar alcohols.

The hydrogenolysis mixture is then removed from the hydrogenolysis reactor. If the hydrogenolysis catalyst is present in dispersed particle form, it is at least in part removed from the reactor together with the hydrogenolysis mixture, and it may be recovered for instance by means of filtration and reinserted in the hydrogenolysis reactor, eventually after being regenerated.

The hydrogenolysis mixture is then separated into at least a low boiling mixture comprising water, ethylene glycol and propylene glycol, and a high boiling mixture, comprising water, glycerol and eventually lactic acid and unreacted sugar alcohols.

Even if any methods known in the art and still to be invented may be used for the hydrogenolysis mixture separation, the preferred way is thermal evaporation. Preferably, the evaporation is conducted at a temperature between 100° C. and 140° C. and at a pressure between 30 mbar and 200 mbar, more preferably at a temperature of 120° C. and at a pressure of 50 mbar.

According to the present disclosure, the high boiling mixture is then inserted in a reforming reactor and contacted with a reforming catalyst at reforming conditions promoting the reforming of the organic compounds in the high boiling mixture. In this specification, by reforming it is meant the overall reaction of an organic compound and water to yield a reforming liquid product and a reforming gas product. The reforming liquid product is in a liquid state at a temperature of 25° C. and a pressure of 1 bar. The reforming gas product comprises reforming Hydrogen and may eventually further comprise methane, carbon monoxide, carbon dioxide, and light alkanes such as ethane and propane. The reforming liquid product comprises water and eventually may further comprise intermediate organic products of the reforming reaction.

Reforming of the high boiling mixture may be conducted in gas phase, according to well-known steam reforming.

Preferably, the reforming of the high boiling mixture is conducted under aqueous phase reforming conditions, wherein the high boiling compounds in the high boiling mixture react with water in the liquid state at aqueous reforming conditions in the presence of a reforming catalyst.

The aqueous reforming catalyst comprises preferably a supported metal selected from the group of Pt, Ru, Re, Pd, Rh, Ni and Co, or combination thereof. The catalyst support may comprise alumina, activated carbon, silica, zeolite, titania, zirconia and ceria, or a combination thereof.

The aqueous reforming reaction is preferably conducted at a reforming temperature promoting the conversion of all, or substantially all, the organic compounds in the high boiling mixture. The aqueous reforming temperature may be between 100° C. to 400° C., preferably between 150° C. to 350° C., and most preferably between 200 to 300° C.

The aqueous reforming reaction is conducted at a reforming pressure which is preferably a reforming pressure sufficient to keep at least a portion of the high boiling compounds in the high boiling polyols mixture in the liquid state at the reforming temperature. The reforming pressure is obtained by means on an inert gas, that is a gas that does not take part to the reforming reaction. Preferably, the inert gas is N₂.

The reforming reaction is preferably conducted in a continuous or semi-continuous mode, wherein the high boiling mixture is inserted in the reforming reactor and/or the reforming products are removed from the reactor continuously or semi-continuously. The continuous or semi-continuous reforming reaction may be characterized by a reforming liquid hourly space velocity of 0.1 to 10 h⁻¹, preferably of 0.5 to 10 h⁻¹, more preferably of 1 to 10 h⁻¹, even more preferably of 2 to 10 h⁻¹, yet even more preferably of 4 to 8 h⁻¹, most preferably of 5 to 7 h⁻¹. The continuous or semi-continuous reforming reaction may be conducted in a CSTR reactor, the catalyst being preferably present in particle form and dispersed in the high boiling mixture in the presence of mechanical agitation. A preferred continuous or semi-continuous reforming configuration the fixed bed reactor, even if the reforming reaction may be conducted also in a fluidized bed reactor. The high boiling mixture, the reforming catalyst and the inert gas may be introduced in the reforming reactor separately from different inlets or may be premixed before the insertion in the reactor.

The reforming hydrogen is reused for supplying at least a portion of the hydrogen needed in the hydrogenation step or the hydrogenolysis step, or both the steps. An external hydrogen source may be used for supplying the remnant portion of the needed Hydrogen.

When the reforming hydrogen is used for supplying the hydrogenation step, the percent ratio of the amount of reforming Hydrogen in the hydrogenation step to the total amount of Hydrogen in the hydrogenation step is greater than a value selected from the group consisting of 10%, 30%, 50%, 60%, 70%, 80%, 90%, and 95%, the remnant portion being supplied by the external hydrogen source.

When the reforming hydrogen is used for supplying the hydrogenolysis step, the percent ratio of the amount of reforming Hydrogen in the hydrogenolysis step to the total amount of Hydrogen in the hydrogenolysis step is greater than a value selected from the group consisting of 10%, 30%, 50%, 60%, 70%, 80%, 90%, and 95%, the remnant portion being supplied by the external hydrogen source.

In a preferred embodiment, all the hydrogen needed in the hydrogenation and hydrogenolysis reaction is supplied by the reforming of the high boiling polyols mixture. The hydrogenation step is preferably conducted in hydrogenation conditions to convert all or substantially all the sugars in the liquid sugar stream to the hydrogenated mixture, that is with a hydrogenation conversion yield of 100% or substantially of 100%. In the disclosed process, the hydrogenolysis step is preferably conducted under suitable hydrogenolysis conditions to produce an amount of high boiling compounds sufficient to generate enough reforming hydrogen to supply all the conversion process. Therefore, in the preferred embodiment, the disclosed process for producing the light polyols mixture is hydrogen self-sufficient. As evident from the experimental section, the amount of high boiling compounds produced in the hydrogenolysis step may be varied and controlled by suitable choice of hydrogenolysis parameters, such as for instance the hydrogenolysis temperature and pH.

In a preferred embodiment, the hydrogenolysis conditions are selected for controlling the amount of glycerol in the hydrogenolysis mixture. The percent amount of glycerol in the hydrogenolysis mixture may be greater than a value selected from the group consisting of 5% to 40%, 10% to 30%, 15% to 20% by weight on a dry matter basis.

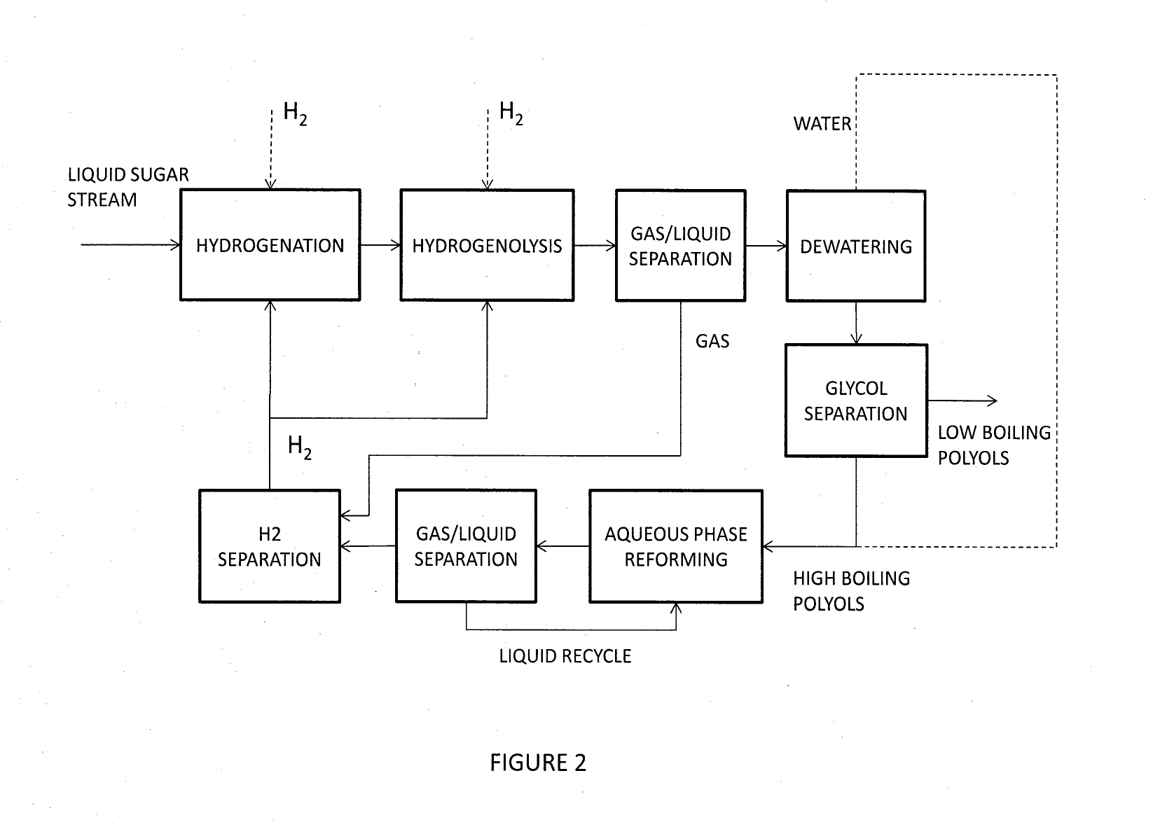

In another preferred embodiment of the disclosed process, represented schematically in FIG. 2, after the hydrogenolysis step the hydrogenolysis mixture is removed from the hydrogenolysis reactor together with unreacted hydrogen and hydrogenolysis gas products and the subjected to a gas/liquid separation step, preferably at a temperature lower than the hydrogenolysis temperature, more preferably at temperature lower than 100° C., most preferably at a temperature lower than 50° C. The unreacted hydrogen and hydrogenolysis gas products may be sent to a first Hydrogen separation step, where hydrogen is purified and then recycled in the hydrogenation and/or hydrogenolysis reaction.

A portion of the water of the hydrogenolysis mixture may be then removed by means of a dewatering step. Dewatering may be done by thermal evaporation or by filtration. Preferably, the dry matter content of the dewatered glycols mixture is a value in the range of 40% to 95%, more preferably of 50 to 90% even more preferably of 60% to 85%, and most preferably of 70% to 80%.

After separating the hydrogenolysis mixture into at least the low boiling mixture and the high boiling mixture, water may be added to the high boiling mixture before reforming step to reach a dry matter of at least 30%, more preferably at least 50% and most preferably at least 60%. Preferably, the added water is water recycled from the dewatering step.

In an embodiment, the pH of the high boiling mixture may be varied before subjecting the, for instance by adding hydrochloric acid, to reach a pH which is preferably between 7 and 9.

After reforming step, reforming gas products and reforming liquid products are separated in a gas/liquid separation step. Gas products enter a second Hydrogen separation step, so as to purify the reforming hydrogen and then used in the hydrogenation and/or hydrogenolysis reaction. Preferably, the first Hydrogen separation step and the second Hydrogen separation are conducted in the same apparatus.

Reforming liquid products may comprise unreacted heavy polyols or intermediate reforming products and may be reinserted in the reforming reactor and further subjected to the reforming step.

### Use

An ethylene glycol stream and a propylene glycol stream may be separated from the low boiling polyols mixture by means of any process known in the art and still to be invented, preferably by means of distillation. Optionally, other streams are produced in the separation.

The propylene glycol stream comprises propylene glycol, and may further comprise small amount of ethylene glycol or other low boiling polyols.

The ethylene glycol stream comprises a plurality of diols, wherein ethylene glycol is the main component, as the amount of ethylene glycol, expressed as molar percent with respect to the plurality of diols, is preferably greater than 80%. In preferred embodiments, the molar amount of ethylene glycol is greater than 85%, being greater than 90% more preferable, greater than 95% even more preferable and greater than 98% the most preferable value.

In an embodiment, the ethylene glycol stream further comprises at least one diol selected from 1,2-Propylene glycol, 1,2-Butanediol and 1,2-Pentanediol.

In a preferred embodiment, the ethylene glycol stream comprises 1,2 Propylene glycol, and the percent molar amount of 1,2 Propylene glycol with respect to the plurality of diols is preferably less than 15%, more preferably less than 12%, even more preferably less than 10%, even yet more preferable less than 7%, even yet more preferable less than 5%, most preferably less than 3%, being less than 2% the even most preferred value.

In another preferred embodiment, the ethylene glycol stream comprises 1,2-Butanediol, and the percent amount of 1,2-Butanediol with respect to the plurality of diols is preferably less than 10%, more preferably less than 8%, even more preferably less than 5%, even yet more preferable less than 3%, most preferably less than 2%, being less than 1% the even most preferred value.

In a preferred embodiment, the ethylene glycol stream comprises 1,2-Pentanediol, and the percent amount of 1,2-Pentanediol with respect to the plurality of diols is preferably less than 5%, more preferably less than 4%, even more preferably less than 3%, even yet more preferable less than 2% and most preferably less than 1%.

Even if the ethylene glycol stream may comprise only one 1,2-diol, more preferably it comprises two 1,2-diols, even more preferably it comprises three 1,2-diols. Most preferably, the ethylene glycol stream comprises 1,2-Propylene glycol, 1,2-Butanediol and 1,2-Pentanediol.

The ethylene glycol stream may be used to produce a polyester resin.

A first preferred method to produce the polyester resin is the ester process, comprising an ester interchange and a polycondensation. Basically, the diols of the plurality of diols are reacted with a dicarboxylic ester (such as dimethyl terephthalate) in an ester interchange reaction, which may be catalyzed by an ester interchange catalyst. As an alcohol is formed in the reaction (methanol when dimethyl terephthalate is employed), it may be necessary to remove the alcohol to convert all or almost all of the reagents into monomers. Then monomers undergo polycondensation and the catalyst employed in this reaction is generally an antimony, germanium or titanium compound, or a mixture thereof. The ester interchange catalyst may be sequestered to prevent yellowness from occurring in the polymer by introducing a phosphorus compound, for example polyphosphoric acid, at the end of the ester interchange reaction.

A second preferred method to produce the polyester resin is the acid process, comprising a direct esterification and a polycondensation. Basically, the diols of the plurality of diols are reacted with an acid (such as terephthalic acid) by a direct esterification reaction producing monomer and water, which is removed to drive the reaction to completion. The direct esterification step does not require a catalyst. Similarly to the ester process, the monomers then undergo polycondensation to form polyester.

In both method, the polyester may be further polymerized to a higher molecular weight by a solid state polymerization, which is particularly useful for container (bottle) application.

In a preferred embodiment, at least 85% of the acid moieties of the polyester are derived from terephthalic acid or its dimethyl ester.

### EXAMPLES

### Liquid Sugar Stream Preparation

Two different wheat straw feedstocks and one Arundo Donax feedstock were used as starting ligno-cellulosic feedstock to produce three sugars liquid streams (stream 1 and stream 2 from wheat straw and stream 3 from Arundo Donax) used in the catalytic conversion experiments.

Each starting ligno-cellulosic feedstock was subjected to a pre-soaking step in water at a temperature of 130° C. for 30 minutes at a liquid-solid ratio of 5:1.

Presoaked ligno-cellulosic feedstock was subjected to a soaking step and the soaked feedstock was separated by means of a press to produce soaking liquid and a soaked solid stream containing the soaked biomass. The soaked solid stream was subjected to steam explosion to create a steam exploded stream. Liquids were separated from the steam exploded stream by means of a press and added to the soaking liquid. In Table 1 the parameters used in soaking step and steam explosion are reported.

TABLE 1
Parameters used in soaking step and steam explosion.

| | Soaking step | | Steam explosion | |
|---|---|---|---|---|
| | Temperature | Time | Temperature | Time |
| | (° C.) | (minutes) | (° C.) | (minutes) |
| Wheat | 155 | 65 | 190 | 4 |
| straw | | | | |
| Arundo | 155 | 155 | 195 | 4 |
| Donax | | | | |

The soaking liquid was subjected to a solid separation step to remove solids, by means of centrifugation and macro filtration (bag filter with filter size of 1 micron). Centrifugation was performed by means of a Alfa Laval CLARA 80 centrifuge at 8000 rpm. A clarified liquid was separated from suspended solids.

The clarified liquid was then subjected to a first nano-filtration step by means of a Alfa Laval 3.8″ equipment (membrane code NF3838/48), which splits the input stream into two streams, the retentate and the permeate. Nano-filtration was performed according to the following procedure.

Permeate flow stability was checked by means of flushing with de-mineral water, at the temperature of 50° C. and 10 bar. Flow rate of the permeate was measured. An amount of 1800 liter of clarified liquid were inserted in the feed tank. Before filtration, the system was flushed for 5 minutes, without pressure, in order to remove the water. The system was set at the operating conditions (pressure: 20 bar, temperature: 45° C.). Retentate stream was recycled in the feed tank and permeate stream was dumped. The test was run until the volume of liquid in the feed tank was reduced up to 50% of the initial soaked liquid volume, corresponding to 900 liters of permeate and 900 liters of retentate. The previous procedure produced a first nano-filtered retentate e a first nano-filtered permeated.

The first retentate liquid was diluted by adding a volume of water corresponding to 50% of its volume and subjected to a second first nano-filtration step, according to the same procedure used in the first nano-filtration step.

The second nano-filtration produced a second nano-filtered permeate and a purified liquid stream.

The purified liquid stream was subjected to a decationization step to produce a decationized liquid stream having a reduced amount of salts, by inserting the purified liquid stream in a column containing an ionic exchange resin (Relite EXC14) at a flow rate of 240 l/h and at the temperature of 25° C. Decationization was performed at a contact time of 3.5 BVH (Bed Volume per Hour).

Each decationized liquid stream was subjected to hydrolysis in a continuous reactor at the conditions reported in Table 2.

TABLE 2
Hydrolysis conditions of the three decationized liquid streams

| | | | Residence time |
|---|---|---|---|
| | pH | Temperature (° C.) | (minutes) |
| decationized liquid stream 1 | 1.75 | 150 | 8.3 |
| decationized liquid stream 2 | 1.34 | 146 | 4.3 |
| decationized liquid stream 3 | 1.33 | 145 | 4.2 |

Each hydrolyzed liquid stream was subjected to a purification step by means of chromatography to produce a corresponding liquid sugar stream. The hydrolyzed liquid stream was inserted in a chromatographic column containing a resin (DIAION UBK 530) at a temperature of 50° C., a flow rate of 601/h and a contact time of 0.5 BVH.

The sugar content of each hydrolyzed liquid stream is reported in the corresponding conversion experiment. The streams may comprise other compounds, which are not relevant for the demonstration of the disclosed conversion process, therefore not reported in the table.

### Example 1

### Batch Hydrogenation Reaction

A volume of 150 ml of stream 1 and an amount of 1.25 g of a 2% Ru/C catalyst (Johnson Matthey Extrudate type 642, grinded to powder form prior to the use), were inserted in a 300 ml stainless steel batch reactor (Parr Instrument). The reactor was sealed, flushed with nitrogen and, finally, pressurized with hydrogen at a pressure of 20 bar at a temperature of 25° C. The reactor was heated to 85° C. in 30 minutes, then kept at 85° C. for 30 minutes and cooled down till 25° C. in 35 minutes. The reaction mixture was separated from the catalyst by filtration on a 0.22 μm PTFE filter and analyzed by means of HPLC. Compositions of the liquid sugar stream 1 and the hydrogenated mixture are reported in the table 3.

TABLE 3
Composition of the liquid sugar stream 1 and
the hydrogenated mixture of example 1.

| | Liquid sugar stream 1 | |
|---|---|---|
| | composition (g/l) | Hydrogenated mixture (g/l) |
| Glucose | 1.39 | 0.92 |
| Arabinose | 1.11 | n.d. |
| Xylose | 35.58 | 8.52 |
| Sorbitol | n.d. | 0.43 |
| Arabitol | n.d. | 2.25 |
| Xylitol | n.d. | 24.2 |

### Example 2

### Batch Hydrogenation Reaction

A volume of 150 ml of stream 2 and an amount of 1.25 g of a 2% Ru/C catalyst (Johnson Matthey Extrudate type 642, grinded to powder form prior to the use), were inserted in a 300 ml stainless steel batch reactor (Parr Instrument). The reactor was sealed, flushed with nitrogen and, finally, pressurized with hydrogen at a pressure of 20 bar at a temperature of 25° C. The reactor was heated to 100° C. in 30 minutes, then kept at 100° C. for 60 minutes and cooled down till 25° C. in 35 minutes. The reaction mixture was separated from the catalyst by filtration on a 0.22 rpm PTFE filter and analyzed by means of HPLC. Compositions of the liquid sugar stream 2 and the hydrogenated mixture are reported in the table 4.

TABLE 4
Composition of the liquid sugar stream 2 and the
hydrogenated mixture of example 2

| | Liquid sugar stream 2 | |
|---|---|---|
| | composition (g/l) | Hydrogenated mixture (g/l) |
| Glucose | 3.16 | n.d. |
| Arabinose | 1.57 | n.d. |
| Xylose | 48.8 | n.d. |
| Sorbitol | n.d. | 2.04 |
| Arabitol | n.d. | 4.16 |
| Xylitol | n.d. | 40.6 |

### Example 3

### Continuous Hydrogenation Reaction

A stainless steel tubular reactor (h 40 cm, i.d. 2 cm) was filled with a 2% Ru/C catalyst (Johnson Matthey Extrudate type 642) and glass beads. The catalytic bed was composed of an upper layer of 4 cm of glass beads (Ø=1.5 mm), a catalyst layer of 25 cm (catalyst extrudate grinded and sieved, 0.6<Ø<1.2 mm) and a bottom layer of 11 cm of glass beads. The reactor was purged with nitrogen and pressurized with hydrogen at a pressure of 60 bar at room temperature. Hydrogen flow was subsequently set to 37 ml/min. The reactor was then heated to 110° C. in 40 minutes and, once the reaction temperature was attained, liquid sugar stream 3 was continuously fed via an HPLC pump at 0.5 ml/min over the catalyst bed, in order to have a liquid hourly space velocity of about 1.5 h⁻¹. Samples of the reaction solution were constantly withdrawn from a collecting tank and analyzed by means of HPLC. Compositions of the liquid sugar stream 3 and the hydrogenated mixture at 20 h and 50 h are reported in Table 5.

TABLE 5
Compositions of the liquid sugar stream 3 and the
hydrogenated mixture at 20 h and 50 h of example 3

| | Liquid sugar | Hydrogenated solution | Hydrogenated |
|---|---|---|---|
| | stream 3 | (g/l) | mixture (g/l) |
| | composition (g/l) | after 20 h on flow | after 50 h on flow |
| Glucose | 1.75 | n.d. | n.d. |
| Arabinose | 4.36 | n.d. | n.d. |
| Xylose | 43.9 | n.d. | n.d. |
| Sorbitol | n.d. | 1.62 | 1.50 |
| Arabitol | n.d. | 7.28 | 6.62 |
| Xylitol | n.d. | 38.9 | 37.2 |

### Example 4

### Continuous Hydrogenolysis Reaction of a Hydrogenated Mixture

A stainless steel tubular reactor (h 40 cm, i.d. 2 cm) was filled with a nickel-based catalyst and glass beads. The catalytic bed was composed of an upper layer of 4 cm of glass beads (Ø=1.5 mm), a catalyst layer of 25 cm (catalyst extrudate grinded and sieved, 0.6<Ø<1.2 mm) and a bottom layer of 11 cm of glass beads.

The reactor was purged with nitrogen and pressurized with hydrogen at a pressure of 60 bar at room temperature. Hydrogen flow was subsequently set to the desired reaction value of 37 ml/min. The reactor was then heated to 200° C. in 90 minutes and, once the reaction temperature was attained, a solution obtained by the hydrogenation of stream 3 (shown in Example 3), to which was added an amount of 5.3 g/l of NaOH, was fed via an HPLC pump at 0.5 ml/min over the catalyst bed, in order to have a liquid hourly space velocity of about 1.5 h⁻¹. Samples of the reaction solution were constantly withdrawn from a collecting tank and analyzed by means of HPLC. Compositions of the hydrogenated stream 3 and the hydrogenolysed mixture at 20 h and 50 h are reported in Table 5. The composition of the hydrogenated stream 3 was close to the compositions of the samplings of Table 5.

TABLE 6
Compositions of the hydrogenated stream 3 and the
hydrogenolysed mixture at 20 h and 50 h of example 4

| | | Hydrogenolysed | Hydrogenolysed |
|---|---|---|---|
| | Feed | solution (g/l) | solution (g/l) |
| | composition | after 20 h | after 50 h |
| | (g/l) | on flow | on flow |
| Sorbitol | 1.37 | n.d. | n.d. |
| Arabitol | 7.71 | 4.28 | 4.68 |
| Xylitol | 36.7 | 6.85 | 8.23 |
| Ethylene glycol | n.d. | 7.94 | 8.51 |
| 1,2-Propylene | n.d. | 7.37 | 7.80 |
| glycol | | | |
| Glycerol | n.d. | 6.52 | 6.76 |
| Lactic acid | n.d. | 2.38 | 2.55 |

Examples 3 and 4 show how, in a continuous mode, a stream can be treated first by hydrogenation and subsequently by hydrogenolysis, effectively yielding Ethylene glycol and 1,2-Propylene glycol.

### Example 5

### Continuous Hydrogenolysis Reaction of a Synthetic Solution

A stainless steel tubular reactor (h 40 cm, i.d. 2 cm) was filled with a nickel-based catalyst and glass beads. The catalytic bed was composed of an upper layer of 4 cm of glass beads (Ø=1.5 mm), a catalyst layer of 25 cm (catalyst extrudate grinded and sieved, 0.6<Ø<1.2 mm) and a bottom layer of 11 cm of glass beads.

In a first hydrogenolysis test (Test 1) the reactor was purged with nitrogen and pressurized with hydrogen at a pressure of 80 bar at a temperature of 25° C. Hydrogen flow was subsequently set to 150 ml/min. The reactor was then heated to 210° C. in 90 minutes and, once the reaction temperature was attained, a water solution composed by 25 wt % of xylitol and 0.5 wt % of NaOH was fed via an HPLC pump at 0.5 ml/min over the catalyst bed, in order to have a liquid hourly space velocity of about 1.5 h⁻¹. Samples of the polyols mixtures were constantly withdrawn from a collecting tank and analyzed by means of HPLC.

In a second hydrogenolysis test (Test 2) the reactor was purged with nitrogen and pressurized with hydrogen at a pressure of 60 bar at a temperature of 25° C. Hydrogen flow was subsequently set to 37 ml/min. The reactor was then heated to 200° C. in 90 minutes and, once the reaction temperature was attained, a water solution composed by 5 wt % of xylitol and 0.5 wt % of NaOH was fed via an HPLC pump at 0.5 ml/min over the catalyst bed, in order to have a liquid hourly space velocity of about 1.5 h⁻¹. Samples of the polyols mixtures were constantly withdrawn from a collecting tank and analyzed by means of HPLC.

Compositions of the polyols mixtures after 20 hours on stream and compared hydrogenolysis performances of Test 1 and 2 are shown in Table 4.

TABLE 5
Compositions of the polyols mixtures and
hydrogenolysis performances of Test 1 and 2

| | Test 1 | Test 2 |
|---|---|---|
| | 25 wt % xylitol, | 5 wt % xylitol, |
| | 210° C., 80 bar, | 200° C., 60 bar, |
| | LHSV 1.5 h⁻¹, | LHSV 1.5 h⁻¹, |
| | xylitol:NaOH | xylitol:NaOH |
| | (mol:mol) 12:1 | (mol:mol) 2:1 |
| | Polyols mixtures composition (g/l) | |
| Xylitol | 28.2 | 7.85 |
| Ethylene glycol | 44.8 | 9.22 |
| 1,2-propylene glycol | 50.9 | 8.05 |
| Glycerol | 15.4 | 6.22 |
| Arabitol | 2.51 | 3.67 |
| Lactic acid | 8.26 | 2.23 |
| | Hydrogenolysis performances (wt %) | |
| Xylitol conversion yield | 85.2 | 83.7 |
| Ethylene glycol selectivity | 27.5 | 23.4 |
| 1,2-Propylene glycol | 31.3 | 20.4 |
| selectivity | | |
| Glycerol selectivity | 9.44 | 15.6 |
| Arabitol selectivity | 1.54 | 8.56 |
| Lactic acid selectivity | 5.07 | 4.98 |

### Example 6

### Aqueous Phase Reforming of High Boiling Polyols

In the paper “Production of hydrogen by aqueous-phase reforming of glycerol” by Z. Tian et al. (International Journal of Hydrogen Energy 33 (2008) 6657-6666), several examples of glycerol conversion to hydrogen via the APR process are reported.

In particular, the experiments were carried out in a tubular reactor (h 58 cm, i.d. 3 cm), containing a volume of 5 ml of the chosen catalyst. Reaction is performed at 230° C., 32 bar and an LHSV of 8.4 h⁻¹, using a 10 wt % solution of glycerol as feed.

First, several metal-based catalysts supported on alumina were tested. Catalysts were prepared by incipient wetness impregnation, starting from water-soluble precursor salts. As it is shown in Table 5, the best results were obtained with the Pt/Al₂O₃ catalyst, considering the product of glycerol conversion to gas and hydrogen produced in the gas phase.

TABLE 5
Comparison of various supported metal catalysts
in the APR of a glycerol solution.

| | Carbon | H₂ amount in the | |
|---|---|---|---|
| | conversion | gas phase | H₂ yield rate |
| Catalyst | to gas (%) | (mol %) | (μmol · min⁻¹ · g_{CAT}⁻¹) |
| Pt/Al₂O₃ | 18.9 | 69.7 | 572.2 |
| Ni/Al₂O₃ | 15.8 | 59.0 | 167.7 |
| Co/Al₂O₃ | 21.0 | 40.9 | 102.2 |
| Cu/Al₂O₃ | 2.0 | 94.5 | 409.8 |

Once the best active phase was defined, the influence of the catalyst carrier was investigated. Several platinum-supported catalyst were prepared and compared.

TABLE 6
Comparison of various platinum supported catalysts
in the APR of a glycerol solution.

| | Carbon | H₂ amount in the | |
|---|---|---|---|
| | conversion | gas phase | H₂ yield rate |
| Catalyst | to gas (%) | (mol %) | (μmol · min⁻¹ · g_{CAT}⁻¹) |
| Pt/Al₂O₃ | 18.9 | 69.8 | 572.2 |
| Pt/SiO₂ | 10.8 | 71.8 | 369.4 |
| Pt/AC | 17.2 | 69.6 | 307.7 |
| Pt/MgO | 13.8 | 79.9 | 431.9 |
| Pt/HUSY | 22.0 | 71.8 | 337.0 |
| Pt/SAPO-11 | 13.3 | 72.8 | 222.1 |

As it is shown in Table 6, alumina seems to be the best performing carrier for platinum catalyst in the APR of glycerol.

### Hydrogen Reactions Balances

### The Overall Reactions Involved in the Conversion of a Xylose Stream to the Polyols Mixture are the Following:

### Hydrogenation:

C₅H₁₀O₅(Xylose)+H₂→C₅H₁₂O₅(Xylitol) (Reaction 1)

### Hydrogenolysis:

C₅H₁₂O₅(Xylitol)+2H₂→C₂H₆O₂(Ethylene glycol)+C₃H₈O₂(1,2-Propylene glycol)+H₂O (Reaction 2)

C₅H₁₂O₅(Xylitol)+H₂→C₂H₆O₂(Ethylene glycol)+C₃H₈O₃(Glycerol) (Reaction 3)

C₅H₁₂O₅→C₂H₆O₂(Ethylene glycol)+C₃H₆O₃(Lactic acid) (Reaction 4)

C₅H₁₂O₅(Xylitol)→C₅H₁₂O₅(Arabitol) (Reaction 5)

The previous hydrogenolysis reactions occur simultaneously

1. Overall Hydrogen Balance of Example 5

Considering that only Reactions 1 to 3 are hydrogen consuming, the respective and overall hydrogen consumption in the production of the polyols mixture are resumed in Table 7.

TABLE 7
Overall Hydrogen consumption of example 4

Moles of H₂ consumed

| | Test 1 | Test 2 |
|---|---|---|
| Reaction 1 | 1.67 | 0.33 |
| Reaction 2 | 1.34 | 0.21 |
| Reaction 3 | 0.17 | 0.07 |
| Overall | 3.18 | 0.61 |

The high-boiling compounds composing the polyols mixture are xylitol, arabitol, glycerol and lactic acid. The high-boiling compounds can be treated by Aqueous Phase Reforming (APR), in order to produce hydrogen.

The APR reactions of the four compounds are:

C₅H₁₂O₅(Xylitol)+5H₂O→5CO₂+11H₂ (Reforming reaction 1)

C₅H₁₂O₅(Arabitol)+5H₂O→5CO₂+11H₂ (Reforming reaction 2)

C₃H₈O₃(Glycerol)+3H₂O→3CO₂+7H₂ (Reforming reaction 3)

C₃H₆O₃(Lactic acid)+3H₂O→3CO₂+6H₂ (Reforming reaction 4)

the respective and overall amount of hydrogen which may be obtained by APR is reported in Table 8.

TABLE 8
Amount of reforming hydrogen of the high boiling
compounds of Test 1 of example 4.

Moles of H₂
produced

| | Test 1 | Test 2 |
|---|---|---|
| Reforming reaction 1 | 2.04 | 0.57 |
| Reforming reaction 2 | 0.18 | 0.47 |
| Reforming reaction 3 | 1.17 | 0.26 |
| Reforming reaction 4 | 0.55 | 0.15 |
| Overall | 3.94 | 1.45 |

Therefore, the amount of Hydrogen which may be generated by aqueous phase reforming of the high boiling compounds present in the polyols mixture is greater than the amount of hydrogen needed for converting the solution of xylose to the polyols mixture.

### Example 7

### Recovery of High Boiling Polyols Mixture

The polyols mixture of example 4 (table 6) was separated in a low boiling polyols mixture and a high boiling polyols mixture.

First, the hydrogenolysed mixture was dewatered by means of thermal evaporation at a pressure of 0.6 bar and a temperature of 70° C., to reach a water content by weight of 30%. The dewatered polyols mixture was separated by means of a high vacuum thermal evaporator at 10 mbar and 85° C. in a low boiling polyols mixture and a high boiling polyols mixture.

The compositions of the two polyols mixtures are reported in table 9.

TABLE 9
Compositions of the low boiling polyols mixture
and the high boiling polyols mixture

| | |
|---|---|
| Low boiling | High boiling |
| polyols mixtures | polyols mixtures |

composition (g/l)

| | | |
|---|---|---|
| Xylitol | n.d. | 531.0 |
| Ethylene glycol | 451.3 | 4.11 |
| 1,2-propylene glycol | 413.7 | 3.77 |
| Glycerol | n.d. | 327.1 |
| Arabitol | n.d. | 151.0 |
| Lactic acid | n.d. | 12.33 |
| Water content (% wt) | 11.5 | 0 |

### Example 8

### Aqueous Phase Reforming of High Boiling Polyols

A stainless steel tubular reactor (h 40 cm, i.d. 2 cm) was filled with 5 wt % Pt/Al₂O₃ catalyst (purchased from Sigma Aldrich) and SiC pellets. The catalytic bed was composed of an upper layer of 4 cm of SiC pellets (Ø=2 mm), a catalyst layer of 25 cm, corresponding to about 30 ml (catalyst extrudate grinded and sieved, 0.6<Ø<1.2 mm) and a bottom layer of 11 cm of SiC pellets. The reactor was then purged with nitrogen and pressurized with hydrogen. Catalyst was activated by flowing 10% H₂ in N₂ at 250° C. for 2 hours with a 30 ml/min gas flow.

In a first aqueous phase reforming experiment, the high boiling polyols mixture was pre-diluted with de-ionized water to a dry matter of about 10 wt %. The reactor was heated at 225° C. and pressurized with nitrogen at 30 bar. Nitrogen was subsequently flown at 30 ml/min. Once both reaction temperature and pressures were attained, the 10 wt % solution thus produced was fed via an HPLC pump at 3.0 ml/min over the catalyst bed, in order to have a liquid hourly space velocity of about 3.0 h⁻¹. Samples of the liquid mixtures were constantly withdrawn from a collecting tank and analyzed by means of HPLC, while the gas phase was directly fed to a GC analyzer, equipped with a sampling valve.

In a second aqueous phase reforming experiment, the high boiling polyols mixture was pre-diluted with de-ionized water to a dry matter of about 10 wt %. The reactor was heated at 225° C. and pressurized with nitrogen at 30 bar. Nitrogen was subsequently flown at 30 ml/min. Once both reaction temperature and pressures were attained, the 10 wt % solution thus produced was fed via an HPLC pump at 0.5 ml/min over the catalyst bed, in order to have a liquid hourly space velocity of about 1.0 h⁻¹. Samples of the liquid mixtures were constantly withdrawn from a collecting tank and analyzed by means of HPLC, while the gas phase was directly fed to a GC analyzer, equipped with a sampling valve.

| | First experiment - | Second experiment - |
|---|---|---|
| | 225° C., 30 bar, | 225° C., |
| | LHSV 3.0 h⁻¹ | 30 bar, LHSV 1.0 h⁻¹ |
| High boiling polyols | 99.7 | 44.2 |
| conversion (%) | | |
| Selectivity to H₂ (%) | 65.1 | 95.3 |
| Moles of H₂ produced per | 48.67 | 31.58 |
| hour | | |

1-34. (canceled)

35. A process for producing a low boiling mixture comprising ethylene glycol and propylene glycol, wherein the process comprises the steps of:
a. hydrogenating a liquid sugar stream derived from a ligno-cellulosic biomass feedstock, said liquid sugar stream comprising water and at least a solubilized monomeric sugar, by contacting the liquid sugar stream with a hydrogenation catalyst in the presence of Hydrogen, at a hydrogenation pressure in the range of 30 bar to 150 bar and at a hydrogenation temperature in the range of 50° C. to 200° C., and for a hydrogenation time sufficient to produce a hydrogenated mixture comprising water and at least a sugar alcohol;
b. conducting hydrogenolysis of at least a portion of the hydrogenated mixture, by contacting the at least a portion of the hydrogenated mixture with a hydrogenolysis catalyst in the presence of OH⁻ ions and Hydrogen, at a hydrogenolysis pressure in the range of 40 bar to 170 bar, at a hydrogenolysis temperature and for a hydrogenolysis time sufficient to produce a hydrogenolysis mixture, comprising ethylene glycol, propylene glycol and glycerol;
c. separating at least a portion of the hydrogenolysis mixture into at least the low boiling mixture comprising ethylene glycol and propylene glycol, and a high boiling mixture comprising glycerol;
d. reforming at least a portion of the high boiling mixture, by contacting the at least a portion of the high boiling mixture with a reforming catalyst under reforming conditions and for a reforming time sufficient to produce a reforming gas product comprising reforming Hydrogen,
wherein the Hydrogen of the hydrogenation step and/or the Hydrogen of the hydrogenolysis step comprises at least a portion of the reforming Hydrogen.

36. The process of claim 35, wherein the percent ratio of the amount of reforming Hydrogen in the hydrogenation step to the total amount of Hydrogen in the hydrogenation step is greater than a value selected from the group consisting of 10%, 30%, 50%, 60%, 70%, 80%, 90%, and 95%.

37. The process of claim 35, wherein the percent ratio of the amount of reforming Hydrogen in the hydrogenolysis step to the total amount of Hydrogen in the hydrogenolysis step is greater than a value selected from the group consisting of 10%, 30%, 50%, 60%, 70%, 80%, 90%, and 95%.

38. The process of claim 35, wherein the percent amount of glycerol in the hydrogenolysis mixture is a value in a range selected from the group consisting of 5% to 40%, 10% to 30%, and 15% to 20% by weight on a dry matter basis.

39. The process of claim 35, wherein the hydrogenolysis temperature is a value in a range selected from the group consisting of 150° C. to 240° C., and 190° C. to 220° C.

40. The process of claim 39, wherein the hydrogenolysis pressure is a value in a range selected from the group consisting of 40 bar to 150 bar, 50 bar to 100 bar, and 60 bar to 80 bar.

41. The process of claim 39, wherein the molar ratio of the total amount of the sugar alcohols to the amount of Hydrogen in the hydrogenolysis step is a value in a range selected from the group consisting of 1:2 to 1:10, 1:3 to 1:8, and 1:4 to 1:6.

42. The process of claim 39, wherein the hydrogenolysis step occurs in a batch mode and the hydrogenolysis time is a value in a range selected from the group consisting of 10 minutes to 10 hours, 20 minutes to 8 hours, 30 minutes to 7 hour, 45 minutes to 6 hours, 60 minutes to 4 hours and 90 minutes to 3 hours.

43. The process of claim 39, wherein the hydrogenolysis step occurs in a continuous or semi-continuous mode having a hydrogenolysis liquid hourly space velocity, and the hydrogenolysis liquid hourly space velocity is a value in a range selected from the group consisting of 0.1 to 4 h⁻¹, 0.2 to 3 h⁻¹, 0.5 to 2.5 h⁻¹, and 1 to 2 h⁻¹.

44. The process of claim 39, wherein the hydrogenolysis catalyst is a supported metal catalyst comprising at least a metal selected from the group consisting of Ru, Ni, Cu and Pt, or combinations thereof and the support comprises at least a compound selected from the group consisting of alumina, zirconia and activated carbon, or a combination thereof.

45. The process of claim 35, wherein the hydrogenation temperature is a value in a range selected from the group consisting of 70° C. to 150° C., 85° C. to 130° C., and 100° C. to 120° C.

46. The process of claim 35, wherein the hydrogenation pressure is a value in a range selected from the group consisting of 40 bar to 150 bar, 50 bar to 100 bar, and 60 bar to 80 bar.

47. The process of claim 35, wherein the molar ratio of the total amount of solubilized monomeric sugars to the amount of Hydrogen in the hydrogenation step is a value in a range selected from the group consisting of 1:2 to 1:10, 1:3 to 1:8, and 1:4 to 1:6.

48. The process of claim 35, wherein the hydrogenation step occurs in a batch mode and the hydrogenation time is a value in a range selected from the group consisting of 30 minutes to 240 minutes, 45 minutes to 180 minutes, and 60 minutes to 120 minutes.

49. The process of claim 35, wherein the hydrogenation step occurs in a continuous or semi-continuous mode having a hydrogenation liquid hourly space velocity, and the hydrogenation liquid hourly space velocity is a value in a range selected from the group consisting of 0.2 to 3 h⁻¹, 0.5 to 2.5 h⁻¹, and 1 to 2 h⁻¹.

50. The process of claim 35, wherein the hydrogenation catalyst is a supported metal catalyst comprising at least a metal selected from the group consisting of Ru, Ni and Pt, or combinations thereof, and the support comprises at least a compound selected from the group consisting of alumina, zirconia and activated carbon, or a combination thereof.

51. The process of claim 35, wherein the reforming is conducted in aqueous phase under aqueous phase reforming conditions comprising a reforming temperature, and the reforming temperature is a value in a range selected from the group consisting of 100° C. to 400° C., 150° C. to 350° C., and 200° C. to 300° C.

52. The process of claim 51, wherein the aqueous phase reforming conditions comprise a reforming pressure, and the reforming pressure is selected to maintain at least a portion of the high boiling polyols in the high boiling mixture in a liquid state at the reforming temperature.

53. The process of claim 51, wherein water is added to the high boiling mixture.

54. The process of claim 51, wherein the reforming catalyst is a supported metal catalyst comprising at least a metal selected from the group consisting of Pt, Ru, Re, Pd, Rh, Ni and Co, or combinations thereof, and the support comprises at least a compound selected from the group consisting of alumina, activated carbon, silica, zeolite, titania, zirconia and ceria, or a combination thereof.

55. The process of claim 51, wherein the reforming step occurs in a continuous or semi-continuous mode having a reforming liquid hourly space velocity, and the reforming liquid hourly space velocity is a value in a range selected from the group consisting of 0.1 to 10 h⁻¹, 0.5 to 10 h⁻¹, 1 to 10 h⁻¹, 2 to 10 h⁻¹, 4 to 8 h⁻¹, and 5 to 7 h⁻¹.

56. The process of claim 35, wherein the steps a), b) and d) are conducted in three separate vessels.

57. The process of claim 35, wherein the at least a sugar alcohol comprises a compound selected from the group consisting of xylitol, sorbitol and arabitol, or mixtures thereof.

58. The process of claim 35, wherein the at least a solubilized monomeric sugar comprises a compound selected from the group consisting of xylose, glucose and arabinose, or mixtures thereof.

59. The process of claim 58, wherein the at least a solubilized monomeric sugar comprises xylose and the percent amount of xylose by weight on a dry matter basis in the liquid sugar stream is greater than a value selected from the group consisting of 50%, 70%, 80%, 90% and 95%.

60. The process of claim 35, wherein at least a portion of the ethylene glycol and at least a portion of the propylene glycol in the low boiling mixture are separated from the low boiling mixture to produce an ethylene glycol stream comprising ethylene glycol and a propylene glycol stream comprising propylene glycol.

61. The process of claim 60, wherein the ethylene glycol stream further comprises at least one diol selected from the group consisting of 1,2-Propylene glycol, 1,2-Butanediol and 1,2-Pentanediol.

62. The process of claim 60, wherein the ethylene glycol stream is used for producing a polyester resin.

63. The process of claim 62, wherein the polyester comprises acid moieties and at least 85 mole % of the acid moieties are derived from terephthalic acid or its dimethyl ester.

64. The process of claim 62, wherein the polyester resin is used for producing a polyester bottle.
